(19) United States
(12) Patent Application Publication
(10) Pub. No.: US 2005/0176782 A1
(43) Pub. Date: Aug. 11, 2005

(54) Medicament and method for treating vulodynia

(76) Inventor: W. Jerry Easterling, San Antonio, TX (US)

Correspondence Address: DAVID G. HENRY, 900 Washington Avenue, 7th Floor, P.O. Box 1470, Waco, TX, 77603-1470, US

(21) Appl. No.: 11/027,022

(22) Filed: Dec. 30, 2004

Related U.S. Application Data
(63) Continuation-in-part of application No. 10/083,625, filed on Feb. 26, 2002.

(51) Int. Cl. A61K 31/455

(52) U.S. Cl. 514/355

(57) ABSTRACT

A medicament and associated treatment involving the use of such medicament for the treatment of vulvodynia. The medicament is in the dosage form of a vaginal suppository, and the primary active ingredient is a calcium antagonist Diltiazem Hydrochloride in the preferred embodiment.

### CITATION TO PARENT APPLICATION(S)

This is a continuation-in-part with respect to U.S. patent application Ser. No. 10/083,625, from which priority is claimed pursuant to 35 U.S.C. 120.

### BACKGROUND OF THE INVENTION

1. Field of The Invention

The present invention relates to medical treatments pertaining to vulvodynia.

2. Background Information

Vulvodynia is characterized by unexplained vulvar pain that can cause physical disability, sexual dysfunction, limitation of normal daily activities, and psychological difficulties. There is currently no accepted successful medical treatment for this condition. The etiology of the disease is unknown, and treatment modalities are poorly defined, and the care of these patients is fragmented with generally poor outcomes.

The problem is oftentimes chronic, lasting for years. One patient treated successfully with the medicament proposed in this writing has suffered with the condition for more than twenty-three years.

The medical characterization of vulvodynia includes but is not limed to (1) vulvar vestibulitis, (2) dysesthetic vulvodynia, (3) vulvar dermatoses, (4) cyclic vulvovaginitis, (5) Uro-vulvovaginitis, (6) atrophic vaginitis, (7) lichen sclerosis, (8) lichen planus, (9) vestibulodynia, (10) vulvar disease, and (11) generalized vulvodynia.

Patient complaints include intense burning, stinging, dryness, dyspareunia, dysuria, bleeding, vaginal discharge, itching, scarring, painful defecation, loss of tissue pigmentation, loss of normal anatomic features, loss of clitoris and labia minora, pain associated with touch, including that of clothing, severe pain with intercourse, and vaginal destruction.

One study conducted by the National Institutes of Health indicates that sixteen percent of women have experienced vulvar pain lasting three months or longer (Harlow, B, Stewart, EG, JAMWA 2003; 58 82-88.) Approximately seventy percent of women with vulvodynia are white, have fair complexion, and are of child bearing age. A study published in the Journal of Urology in May 1997 suggested that ten percent of women with interstitial cystitis also have symptoms of vulvodynia.

Treatments for vulvodynia include oral medications such as atihistamines, tricyclic antidepressants, topical estrogens, topical testosterone, and aticonvulsants; physical therapy and biofeedback; interferon intralesional injections; low oxalate diet; oral calcium citrate; laser therapy; and surgery. Laser and surgical treatment complications include hematoma, wound dehiscence, uneven healing, and stenosis of the Bartholin's duct with cyst formation. There is no known cure for vulvodynia.

### SUMMARY OF THE INVENTION

In view of the foregoing, It is an object of the present invention to provide a novel medicament to be used for the treatment of vulvodynia.

It is another object of the present invention to provide a novel medicament and unobvious medicament for the treatment of vulvodynia, which medicament is more effective than existing means for treatment.

In satisfaction of these and related objectives, Applicant's present invention provides the vulvo-vaginal application of a calcium channel blocker agent (preferably in the form of a topically applied gel suspension) and associated methodology for use therof, through the use of which vulvodynia may be effectively, noninvasively, cost effectively, and painlessly treated.

### DETAILED DESCRIPTION OF THE PREFERRED EMBODIMENT

The preferred embodiment of the medicament of the present invention is gel suspension which has demonstrated relief from the symptoms of vulvodynia in as little as ten days of treatment.

In the preferred embodiment, the primary active ingredient of the gel suspension is any calcium channel blocking agent, but preferably nifedipine, a dihydropyridine calcium channel blocker. Other calcium channel blockers that could be utilized include verapamil, a diphenylalkamine, diltiazem, a benzothiazepine calcium channel blocker, and the fast dosium inward channel inhibitor, Bepridil. Calmodulin blocking agents such as trifluoperazine could also be used. The primary objective of the treatment regimen is to render divalent calcium biologically inactive in diseased tissue cells resulting in a tissue remodeling effect along with the relief of pain.

The preferred nifedipine-based suspension is a 2% topical gel applied topically to all affected areas twice a day or a 4% nifedipine topical gel applied topically to affected areas only at bedtime. The choice of treatment regimen is based on drug tolerance and issues of patient convenience. The patient measures 0.5 mL of medication from a 30 Gm glaminate tube, utilizing a paper dosimeter, removes the medication from the dosimeter with the forefinger and gently applies the medication to the affected areas.

The preferred nifedipine-based 2% topical gel formulation follows, with the 4% formulation being identical with the exception of doubling the quantity of nifedipine).

| | |
|---|---|
| Nifedipine 2% Topical Gel | 480 mL |
| Nifedipine, USP | 9.6 Gm |
| Lecithin Soya Granular | 65.6 Gm |
| Isopropyl Myristate, NF | 65.6 Gm |
| Pluronic F127, NF (Poloxamer 407) | 58.6 Gm |
| Water (Purified), USP | 236 Gm |
| Potassium Sorbate, NF | 0.78 Gm |
| Sorbic Acid, NF | 0.35 Gm |

The procedure for preparing the gel is as follows:
Place Nifedipine, Lecithin Soya Granular, Sorbic Acid, and Isopropyl Myristate in a beaker and stir with a dissolver blade until smooth. Add Pluronic F127, Potassium Sorbate, and Water and continue stirring until smooth.
Dispense in 30 Gm glaminate tubes. Store at room temperature and protect medication from direct exposure to light.

Patient progress should be evaluated by the patient's physician every sixty days. The patient should be questioned regarding any side effects such as localized skin irritation, heart rate changes, and blood pressure changes characterized by dizziness.

The inventor believes that upon successful absorption of the nifedipine, that the calcium channel blocking properties of the nifedipine may exert an antivaso-constrictor activity and/or initiate a non-vascular process such as serotonin release or serotonin and histamine receptor blockade. The inventor also believes that after repeated use of the medicament, that a tissue remodeling of diseased tissue occurs, resulting in healthier tissue along with an accompanying relief of pain. The tissue remodeling occurs as a result of the control of the production of fibroblasts, the maturation of fibroblast metaloproteinase, and the interaction of cytokines. The inventor believes that healthy tissue is unaffected by the medicament.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

1. A medicament for the treatment of vulvodynia comprising:
a therapeutic dosage of a calcium channel blocker agent;
a host medium configured for topical application.

2. The medicament of claim 1 wherein said calcium channel blocker agent is Nifedipine.

3. A method for treating vulvodynia comprising the steps of:
selecting a dosage form comprising:
a therapeutic dosage of a calcium channel blocker agent;
a host medium configured for topical application;
administering a combination of said calcium channel blocker suspended in said host medium to vulvar areas of a human patient suffering from vulvodynia until symptoms subside.

4. The method of claim 4 wherein said calcium channel blocker agent is Nifedipine.
